(19) United States
(12) Patent Application Publication
(10) Pub. No.: US 2017/0239442 A1
(43) Pub. Date: Aug. 24, 2017

(54) CATHETER

(71) Applicant: ASAHI INTECC CO., LTD., Nagoya-shi (JP)

(72) Inventor: Masatomo ISHIKAWA, Seto-shi (JP)

(73) Assignee: ASAHI INTECC CO., LTD., Nagoya-shi (JP)

(21) Appl. No.: 15/370,288

(22) Filed: Dec. 6, 2016

(30) Foreign Application Priority Data
Feb. 22, 2016 (JP) 2016-030691

(51) Int. Cl. A61M 25/00; A61M 5/00; A61M 5/145

(52) U.S. Cl. CPC A61M 25/0026; A61M 25/0045; A61M 5/1452; A61M 5/007; A61M 25/007; A61M 2025/004

(57) ABSTRACT

A catheter allowing injection or aspiration of fluid at any desired position along an entire circumference of the catheter. The catheter may include a first hollow body having an inner lumen, and a second hollow body covering an outer periphery of the first hollow body and having an outside lumen between the first hollow body and the second hollow body. The outside lumen is divided by partition walls connected to the first hollow body and the second hollow body to form a plurality of outer lumens. On an outer periphery of the second hollow body, holes are formed for the respective outer lumens along a circumferential direction of the second hollow body. Alternatively, the catheter may include a plurality of tubes disposed on an outer periphery of the hollow body, and may include a hole formed in each of the tubes along a circumferential direction of the hollow body.

### CROSS REFERENCE TO RELATED APPLICATION

This application claims priority to Japanese Application No. 2016-030691 filed on Feb. 22, 2016, the contents of which are incorporated by reference herein in their entirety.

### BACKGROUND

The disclosed embodiments relate to a medical device. Specifically, the disclosed embodiments relate to a catheter that is configured to be inserted into a lumen such as a blood vessel for use.

During a conventional procedure, a physician will insert a catheter into a body lumen such as a blood vessel, and then insert a guide wire into a lumen of a catheter to maneuver the catheter through the body lumen to reach a lesion part. Alternatively, physicians may dispose devices such as a stent and an embolic coil at the lesion part via the lumen of the catheter. Physicians have also injected, using a catheter having a side hole on the surface thereof, a medical liquid or a contrast medium into a blood vessel through the side hole, or have secured blood flow (i.e., aspirated blood) through the side hole when the catheter is left in a blood vessel or the like. A catheter can be more efficient at injecting a contrast medium or the like or securing blood flow if the catheter has side holes on the entire circumference thereof.

For example, Japanese Patent Application Laid-open No. 2006-346183 describes a triple-lumen catheter including a first lumen, a second lumen, and a third lumen (see FIG. 1, and the like), where the third lumen of the triple-lumen catheter has a side hole (see paragraph [0020], FIG. 1, and the like). Japanese Patent Application Laid-open No. 2006-346183 also describes that in the triple-lumen catheter, the third lumen communicates with the outside through the side hole (see paragraph [0019], FIG. 1, and the like).

In addition, Japanese Patent Application Laid-open No. 2006-346183 describes a triple-lumen catheter main body, in which a blood removal lumen has two side holes.

However, in the above-described conventional catheter including a plurality of lumens, no lumen is formed on the entire circumference of the catheter, and thus the position of a side hole formed on a lumen is restricted. Therefore, when a contrast medium or the like is injected or when blood flow is secured, it is difficult to inject a contrast medium or the like or secure blood flow at a desired position along the circumference of the catheter.

As a result, in order to inject a contrast medium or the like or secure blood flow at any desired position along the entire circumference of the catheter, it has been necessary to consider the positioning of a lumen for injecting a contrast medium or the like or securing a blood flow.

### SUMMARY

In view of the above-described problems of the conventional technology, the disclosed embodiments have been devised to provide a catheter capable of injecting a contrast medium or the like or securing blood flow at any desired position along the entire circumference of the catheter.

In order to address the above-described problems, a catheter according to the disclosed embodiments includes a first hollow body that has an inner lumen, and a second hollow body that covers an outer periphery of the first hollow body and has an outside lumen that is formed between the first hollow body and the second hollow body. The outside lumen is divided by a plurality of partition walls connected to the first hollow body and the second hollow body to form the plurality of outer lumens. On an outer periphery of the second hollow body, a hole is formed along a circumferential direction of the second hollow body for each of the outer lumens. In other words, a plurality of holes are formed in the second hollow body within a same longitudinal cross-sectional portion of the catheter so that each of the outer lumens communicates with one of the holes.

That is, on the outer periphery of the second hollow body, a plurality of holes are formed along the circumferential direction of the second hollow so that a contrast medium or the like can be injected from the holes, or blood can be aspirated to secure blood flow through the holes. Here, the second hollow body includes the outside lumen on the outer periphery of the first hollow body. A plurality of partition walls are provided between the first hollow body and the second hollow body to form the outer lumens, and a hole is formed on the outer periphery of the second hollow body for each of the outer lumens. This makes it possible to inject a contrast medium or the like or secure blood flow at any desired position along the entire circumference of the catheter.

The outer lumens may instead be formed by disposing a plurality of tubes along a longitudinal direction of the catheter on the outer periphery of the first hollow body; a hole is formed for each of the tubes. That is, a hole is formed in each of the tubes along a same longitudinal cross-sectional portion of the hollow body. This configuration prevents kinking of the outer lumens. Moreover, by using the tubes to form the outer lumens, it is easier to manufacture the catheter. Thus, it is possible to easily achieve injection of a contrast medium or the like or to secure blood flow at a desired position along the circumference of the catheter.

The tubes may instead be formed by being wound into a helical coil structure on the outer periphery of the first hollow body. This configuration improves the flexibility of the catheter. Moreover, the second hollow body can be easily formed using a conventional twisting machine. Thus, it is possible to even more easily achieve injection of a contrast medium or the like or to secure blood flow at a desired position along the circumference of the catheter.

### BRIEF DESCRIPTION OF THE DRAWINGS

FIGS. 1A and 1B are diagrams each illustrating an overview of a catheter according to the disclosed embodiments. FIG 1A is a plan view and FIG. 1B is a cross-sectional view.

FIG. 2 shows an enlarged view of a distal end of the catheter shown in FIG. 1B.

FIG. 3 is a cross-sectional view along line A-A of FIG. 2.

FIG. 4 is a diagram illustrating an overview of a catheter according to the disclosed embodiments.

FIG. 5 is a cross-sectional view along line B-B of FIG. 4.

FIG. 6 is a diagram illustrating an overview of a catheter according to the disclosed embodiments.

FIG. 7 is a cross-sectional view along line C-C of FIG. 6.

FIG. 8 is a diagram illustrating an overview of a catheter according to the disclosed embodiments.

### DETAILED DESCRIPTION OF EMBODIMENTS

The following is a description of the catheter of the disclosed embodiments with reference to the drawings.

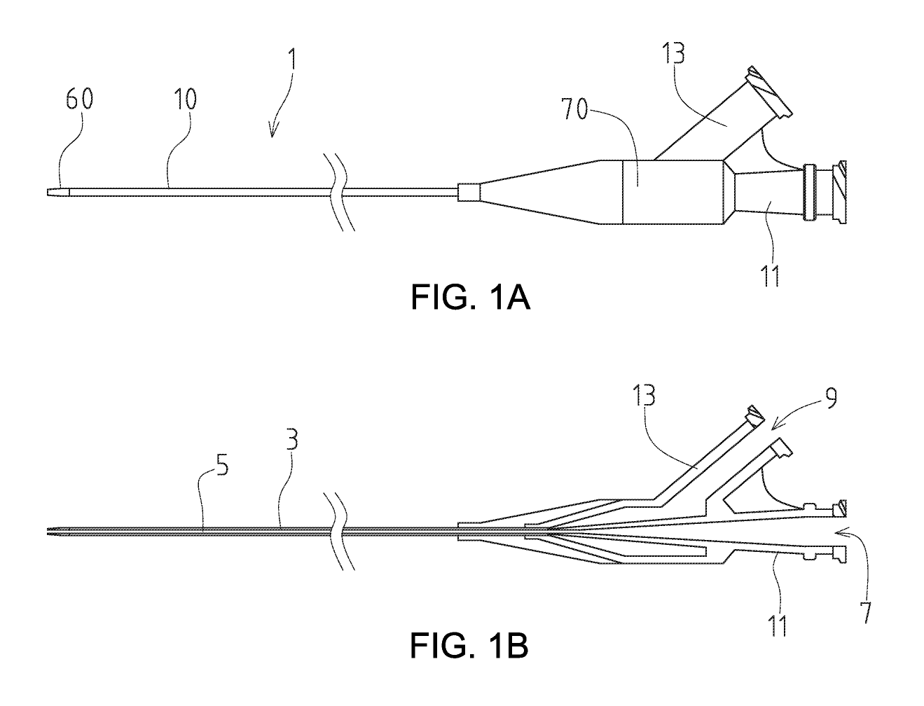

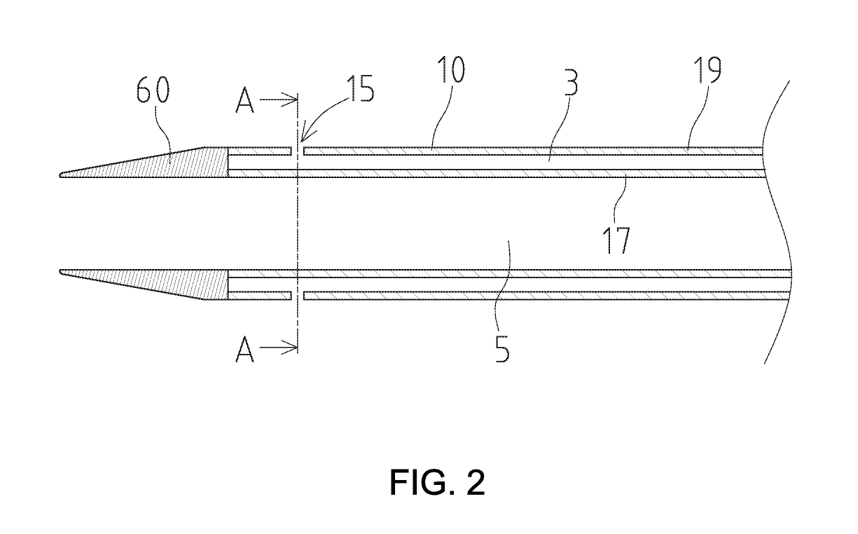

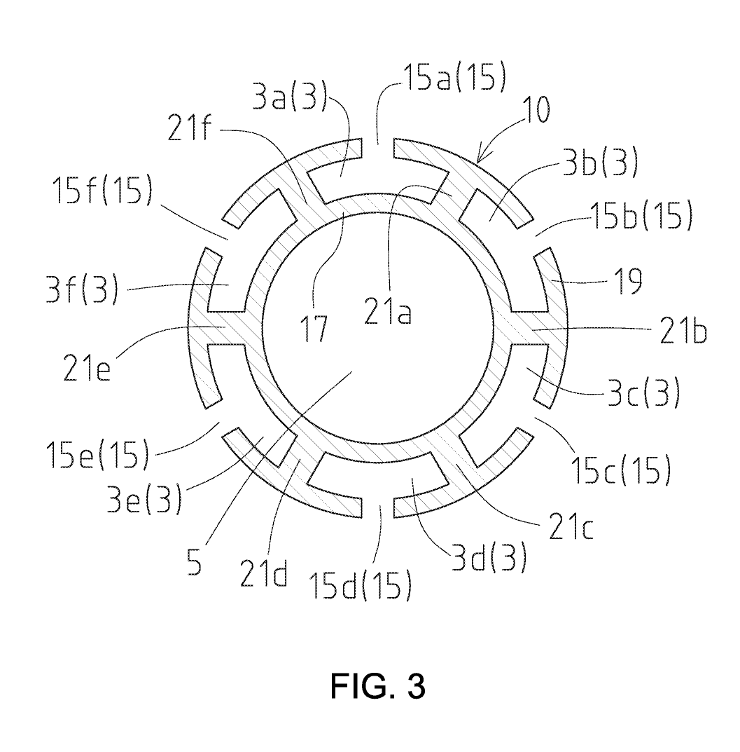

FIGS. 1A and 1B are diagrams each illustrating an overview of a catheter according to the disclosed embodiments. FIG. 1A is a plan view and FIG. 1B is a cross-sectional view. FIG. 2 shows an enlarged view of a distal end of the catheter shown in FIG. 1B, and FIG. 3 is a cross-sectional view along line A-A of FIG. 2.

As illustrated in FIG. 1A, a catheter 1 of the disclosed embodiments includes a catheter shaft 10, a distal end tip 60 provided at a distal end of the catheter shaft 10, and a connector 70 provided at a proximal end of the catheter shaft.

As illustrated in FIG. 1B, the catheter shaft 10 and the distal end tip 60 have a hollow shaped double-lumen structure including an inner lumen 5 in the center of the section and an outside lumen 3 formed on the outer periphery of the first lumen 5 with the same axis. That is, the inner lumen 5 and the outside lumen 3 are concentrically disposed about the longitudinal axis of the catheter 1.

The connector 70 includes a first connector 11 with a first opening 7 through which a guide wire or the like can be inserted, and a second connector 13 with a second opening 9 to which a syringe (not illustrated) for injecting a contrast medium or the like can be connected. The first opening 7 communicates with the inner lumen 5, and the second opening 9 communicates with the outside lumen 3.

Moreover, as illustrated in FIGS. 2 and 3, the catheter shaft 10 includes a first hollow body 17, a second hollow body 19, and partition walls 21a to 21f that are connected to the first hollow body 17 and the second hollow body 19 to form a plurality of outer lumens 3a to 3f (3).

On the outer periphery of the catheter shaft 10, a plurality of holes 15 are formed in the vicinity of a joint between the catheter shaft 10 and the distal end tip 60 (at a distal end of the catheter shaft 10). In the catheter 1 depicted in FIG. 3, six holes 15 in total (holes 15a to 15f) are formed in the circumferential direction of the catheter shaft 10 at an interval of about 60° about the longitudinal axis of the catheter 1.

Here, when a contrast medium or the like is injected from a syringe connected to the opening 9, the contrast medium or the like is diverted through the outer lumens 3a to 3f (3) (see FIG. 3) formed in the catheter shaft 10, and is discharged from the six holes 15a to 15f (15).

Note that a resin material forming the distal end tip 60 is not particularly limited. For example, polyurethane resin may be used because it is relatively flexible and would reduce the risk of damage to blood vessels.

The catheter 1 includes the first hollow body 17 that has the inner lumen 5, and the second hollow body 19 that covers an outer periphery of the first hollow body 17 and has the outside lumen 3 between the first hollow body 17 and the second hollow body 19. Additionally, the outside lumen 3 is divided by a plurality of partition walls 21a to 21f connected to the first hollow body 17 and the second hollow body 19, and on an outer periphery of the second hollow body 19, the hole 15 is formed along a circumferential direction of the second hollow body 19 for each of the outer lumens 3a to 3f. Thus, it is possible to inject a contrast medium or the like at any desired position along the entire circumference of the catheter 1.

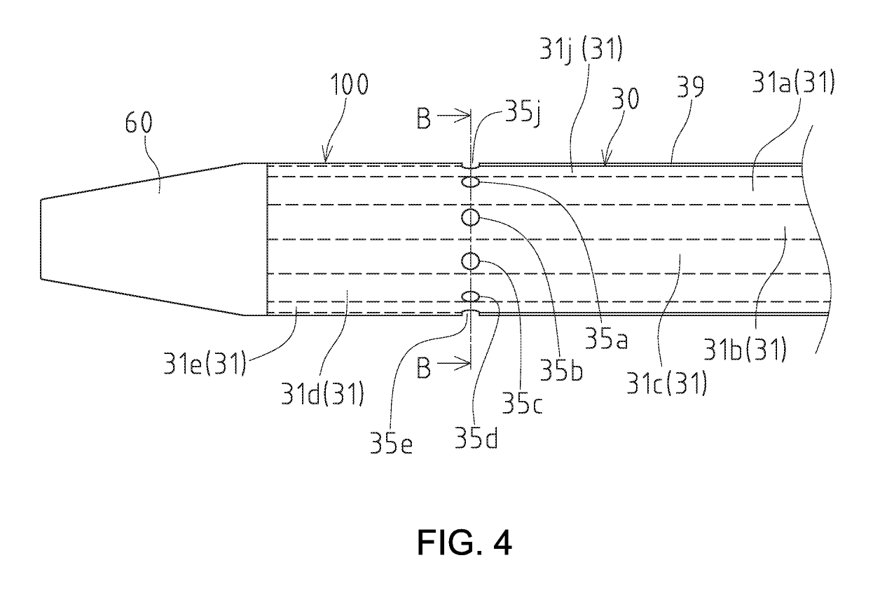

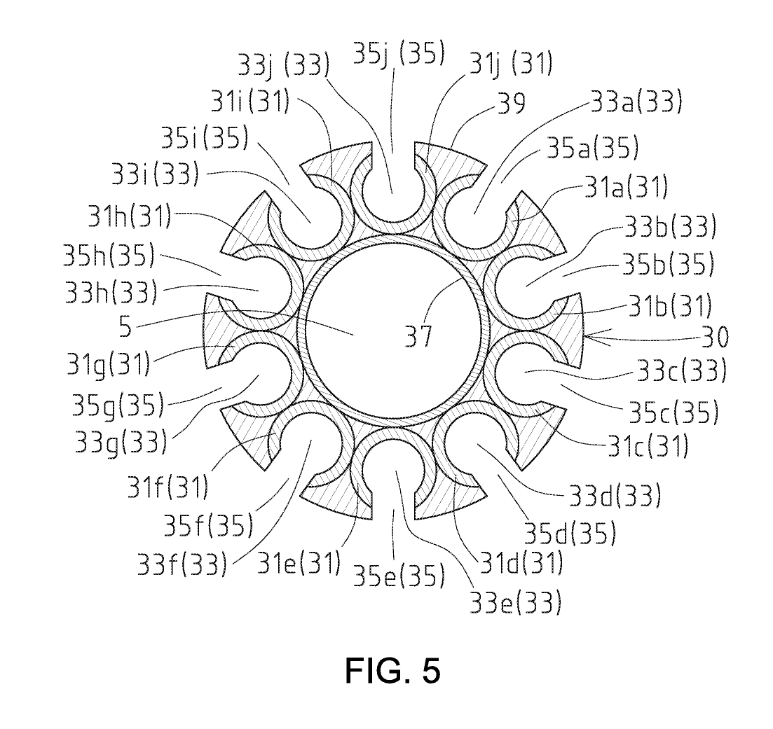

FIG. 4 is a diagram illustrating an overview of a catheter 100, and FIG. 5 is a cross-sectional view along line B-B of FIG. 4. The catheter 100 is different from the above-described catheter 1 in the following point. That is, in the catheter 1, the first hollow body 17, the second hollow body 19, and the plurality of partition walls 21a to 21f form the outer lumens 3a to 3f, and the holes 15a to 15f are formed for the respective outer lumens 3a to 3f. Meanwhile, in the catheter 100 of the second embodiment, a plurality of tube bodies 31 are disposed along a longitudinal direction of the catheter on the outer periphery of a hollow body 37. An outer layer 39 covers an outer periphery of the tube bodies 31, and a hole 35 is formed for each of the tube bodies 31.

As illustrated in FIG. 4, a catheter shaft 30 includes the hollow body 37, the plurality of tube bodies 31 provided on the outer periphery of the hollow body 37, and the outer layer 39 covering the plurality of the tube bodies 31.

On the outer periphery of the catheter 100, a plurality of holes 35 are formed in the vicinity of a joint between the catheter shaft 30 and the distal end tip 60. In the embodiment, 10 holes 35 in total (holes 35a to 35j) are formed in the circumferential direction of the catheter 100 at an interval of about 36° about the longitudinal axis of the catheter 100, as illustrated in FIG. 5.

Here, when a contrast medium or the like is injected from a syringe connected to the opening 9, it is diverted through outer lumens 33a to 33j (33) formed by the tube bodies 31a to 31j (see FIG. 5) formed in the catheter 100, and is discharged from the 10 holes 35a to 35j (35).

Note that the tube bodies 31 are formed of resin, and may be formed of polyether ether ketone (PEEK) resin. The PEEK resin has a melting point of about 330° C., and is known as a resin having a relatively high heat-resistant property for thermoplastic resin, allowing injection molding.

Moreover, the outer layer 39 is also formed of resin, and a polyamide, polyamide elastomer, polyester, polyurethane, and the like can be used, for example, without any limitation thereto.

In the catheter 100, a plurality of tube bodies 31a to 31j (10 tube bodies) are formed on the circumference of the hollow body 37, and the holes 35a to 35j (35) are formed for the respective tube bodies. This prevents kinking of the outer lumens 33a to 33j (33) through which a contrast medium or the like is injected.

Moreover, with the use of the tube bodies 31, the manufacture of the catheter is easier. It is therefore possible to easily achieve injection of a contrast medium or the like or to secure blood flow at any desired position along the entire circumference of the catheter 100.

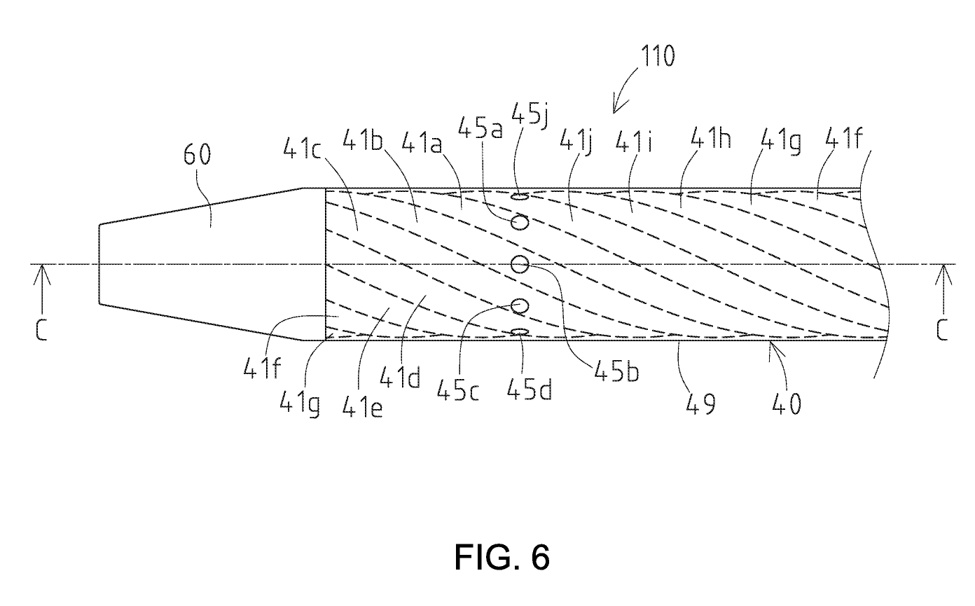

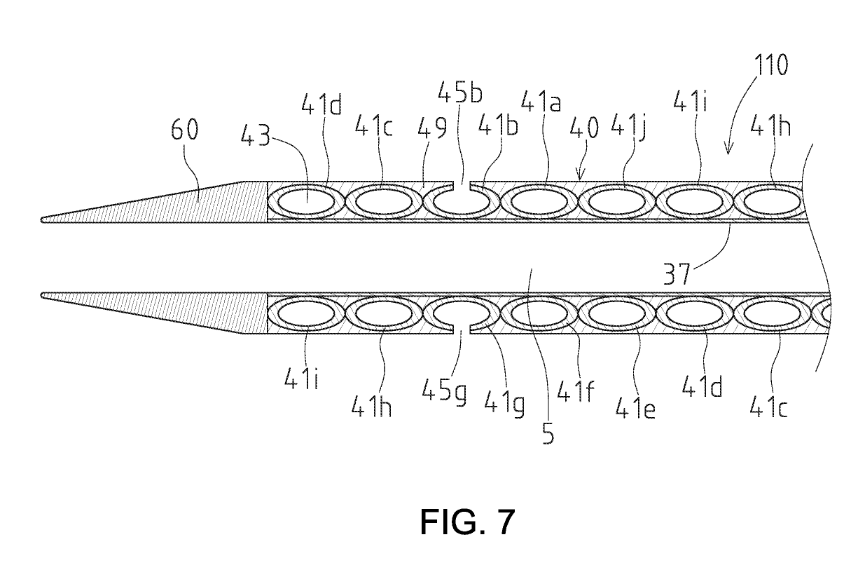

FIG. 6 is a diagram illustrating an overview of a catheter 110 according to the disclosed embodiments, and FIG. 7 is a cross-sectional view along line C-C of FIG. 6. The catheter 110 is different from the above-described catheter 100 in the following point. That is, in the catheter 100, a plurality of tube bodies 31 are disposed along a longitudinal direction of the catheter 100 on the outer periphery of the hollow body 37. Meanwhile, in the catheter 110, a plurality of tube bodies 41 (41a to 41j) are wound into a helical coil structure along a longitudinal direction of the catheter 110 on the outer periphery of the hollow body 37, and a hole 45 (45a to 45j) is formed for each of the tube bodies 41.

As illustrated in FIG. 6, a catheter shaft 40 includes the hollow body 37, a plurality of tube bodies 41 disposed on the outer periphery of the hollow body 37, and an outer layer 49 covering the plurality of the tube bodies 41.

On the outer periphery of the catheter 110, a plurality of holes 45 are formed in the vicinity of a joint between the catheter shaft 40 and the distal end tip 60. In the catheter 110, 10 holes 45 in total (holes 45a to 45j) are formed in the circumferential direction of the catheter 110 at an interval of about 36° about the longitudinal axis of the catheter 110.

Here, when a contrast medium or the like is injected from a syringe connected to the opening 9, it is diverted through the outer lumens 43 formed by the tube bodies 41a to 41j formed in the catheter 110, and is discharged from the 10 holes 45a to 45j (45).

Note that the tube bodies 41 may be formed of polyether ether ketone (PEEK) resin, similarly to the above-described tube bodies 31. Also, for the outer layer 49, the same resin as for the above-described outer layer 39 can be used.

In the catheter 110, the plurality of tube bodies 41 are wound into a helical coil structure on the outer periphery of the hollow body 37. This improves the flexibility of the catheter 110. Moreover, the tube bodies 41 can be easily wound on the outer periphery of the hollow body 37 using a conventional twisting machine. Thus, it is possible to more easily achieve injection of a contrast medium or the like or to secure blood flow at any desired position along the entire circumference of the catheter 110.

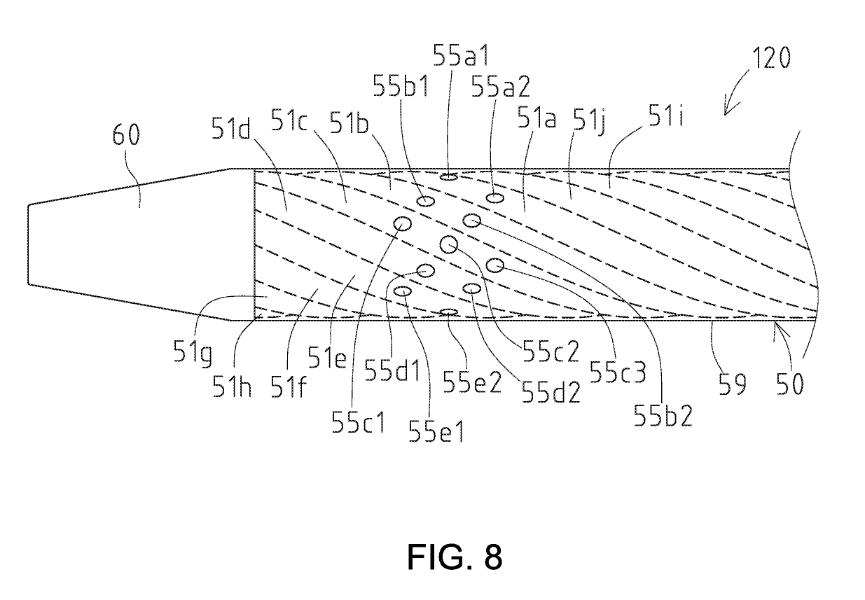

FIG. 8 is a diagram illustrating an overview of a catheter 120 according to the disclosed embodiments. The catheter 120 is different from the above-described catheter 110 in the following point. That is, in the catheter 110, the single hole 45 is formed for each of the tube bodies 41 wound into a helical coil structure. Meanwhile, in the catheter 120, a plurality of holes 55 are formed for each of tube bodies 51 wound into a helical coil structure.

As illustrated in FIG. 8, a catheter shaft 50 includes the hollow body 37 (not shown), a plurality of tube bodies 51 provided on an outer periphery of the hollow body 37, and an outer layer 59 covering the plurality of the tube bodies 51.

Moreover, on the outer periphery of the catheter 120, a plurality of holes 55 (e.g., 55a1, 55a2, 55b1, 55b2, 55c1, 55c2, 55c3, 55d1, 55d2, 55e1, and 55e2) are formed in the vicinity of a joint between the catheter shaft 50 and the distal end tip 60.

Here, when a contrast medium or the like is injected from a syringe connected to the opening 9, it is diverted through outer lumens 53 (not illustrated) formed by the tube bodies 51a to 51j formed in the catheter 120, and is discharged from the holes 55.

Note that the tube bodies 51 may be formed of polyether ether ketone (PEEK) resin, similarly to the above-described tube bodies 31 and tube bodies 41. Also, for the outer layer 59, the same resin as for the above-described outer layer 39 and outer layer 49 can be used.

What is claimed is:

1. A catheter comprising:
a first hollow body that includes an inner lumen; and
a second hollow body that covers an outer periphery of the first hollow body and includes an outside lumen between the first hollow body and the second hollow body,
wherein:
the outside lumen is divided by a plurality of partition walls connected to the first hollow body and the second hollow body to form a plurality of outer lumens, and
on an outer periphery of the second hollow body, a plurality of holes are formed in the second hollow body, the plurality of holes located within a same longitudinal cross-sectional portion of the catheter, each of the outer lumens communicating with one of the holes.

2. A catheter comprising:
a hollow body that includes an inner lumen; and
a plurality of tubes disposed on an outer periphery of the hollow body,
wherein a hole is formed in each of the tubes along a same longitudinal cross-sectional portion of the hollow body.

3. The catheter according to claim 2, wherein a plurality of holes is formed in each of the tubes.

4. The catheter according to claim 2, further comprising:
an outer layer covering the plurality of tubes.

5. The catheter according to claim 2, wherein the tubes are disposed along a longitudinal direction of the catheter.

6. The catheter according to claim 5, further comprising:
an outer layer covering the plurality of tubes.

7. The catheter according to claim 2, wherein the tubes are wound into a helical coil structure on the outer periphery of the hollow body.

8. The catheter according to claim 7, wherein a plurality of holes is formed in each of the tubes.

9. The catheter according to claim 7, further comprising:
an outer layer covering the plurality of tubes.
